(19) United States
(12) Patent Application Publication
(10) Pub. No.: US 2004/0247703 A1
(43) Pub. Date: Dec. 9, 2004

(54) Composition for inhibiting glutamate-mediated neurotoxicity comprising ginsenoside Rg3 or ginsenoside Rh2

(76) Inventors: Hyewhon Rhim, Seoul (KR); Sunoh Kim, Seoul (KR)

Correspondence Address: Pennie & Edmonds LLP, 3300 Hillview Avenue, Palo Alto, CA, 94304, US

(21) Appl. No.: 10/695,501

(22) Filed: Oct. 27, 2003

(30) Foreign Application Priority Data
Oct. 26, 2002 (KR) 10-2002-0065648

(51) Int. Cl. A61K 31/7048; A61K 35/78

(52) U.S. Cl. 424/728; 514/33

(57) ABSTRACT

Disclosed is a composition for inhibiting glutamate-mediated neurotoxicity comprising 20(S)-ginsenoside Rg₃, 20(S)-ginsenoside Rh₂ or the mixture of 20(S)-ginsenoside Rg₃ and 20(S)-ginsenoside Rh₂. The composition of the present invention has an excellent neurotoxicity inhibition effect or nerve cell protection effect. Accordingly, it can be effectively used for treating nervous diseases including cerebral trauma, cerebral apoplexy, cerebral hemorrhage; epilepsy occurred by excessive activation of glutamate receptors; or Huntington's disease.

### FIELD OF THE INVENTION

The present invention relates to a composition for inhibiting glutamate-mediated neurotoxicity, more particularly to a composition for inhibiting glutamate-mediated neurotoxicity comprising 20(S)-ginsenoside Rg₃ and/or 20(S)-ginsenoside Rh₂.

### BACKGROUND OF THE INVENTION

Ginseng (root of Panax ginseng, C. A. Meyer (Araliaceae)) is one of the most frequently used herb medicines not only in Asia but throughout the world. Ingredients having biological efficacies of ginseng include more than 30 kinds of compounds known as ginsenosides or ginseng saponin (Liu, C. X. et al., J. Ethnopharmacol. 36, 27-38, 1996 and Baek, N. I. et al., Planta Med. 1996, 62, 86-87, 1996). Representative examples are ginsenoside-Ra, -Rb₁, -Rb₂, -Rc, -Rd, -Re, -Rf, -Rg₁, -Rg₂, -Rg₃, -Rh₁, -Rh₂ and the like.

Recent studies have reported the benefits on the central nervous system among various properties of ginseng saponin within a living body. Ginseng has been shown to have advantageous effects on the memory and learning capability of animals. Particularly, ginseng has known to relieve cognitive function damage in animals caused by scopolamine, age-associated memory impairment, and memory failure owing to damaged hippocampus nerve cells, which are known to be the most important in memory formation (Hsieh, M. T. et al., Phytother. Res. 14, 375-377, 2000, Wesnes, K. A., Ward et al., Psychopharmacology (Berl) 152, 353-361, 2000 and Zhong, Y. M. et al., Physiol. Behav. 69, 511-525, 2000).

Besides such effects, there have been other reports of ginseng's effects on encephalopathies such as cerebral hemorrhage, cerebral apoplexy and cerebral ischemia. In the case of brain damages caused by external impact, or cerebrovascular occlusion-induced brain damages, the homeostasis of glutamate, which is a representative excitatory neurotransmitter, is destroyed to thereby kill nerve cells because of glutamate-induced neurotoxicity. Several studies show that ginseng treatment to an animal having cerebral ischemia induced by occlusion in the myocardium or forebrain blood vessels can prevent the nerve cells death (Wen, T. C. et al., Acta. Neuropathol. (Berl) 91, 15-22, 1996 and Maffei Facino, R. et al., Planta Med. 65, 614-619, 1999).

Such nerve cell protection effect of ginseng was verified by the fact that one of components of ginseng, ginsenoside Rbi inhibits death of hippocampus nerve cells, which is damaged most severely during the cerebral ischemia, and the glutamate-induced neurotoxicity in a cultured cortical neuron is inhibited by ginsenosides Rb₁ and Rg₃ (Lim, J. H. et al., Neurosci Res. 28, 191-200, 1997, and Kim, Y. C. et al., J. Neurosci. Res. 53, 426-432, 1998).

Despite continued publications on the various benefits of ginseng on the central nervous system, neither the detailed action mechanism nor the various effects of each ginseng component have yet been disclosed.

### SUMMARY OF THE INVENTION

The object of the present invention is to elucidate the glutamate-induced neurotoxicity inhibition mechanism of ginsenosides contained in ginseng and to provide a composition for inhibiting glutamate-mediated neurotoxicity comprising ginsenoside having an excellent inhibition capability against NMDA receptors, which are most important for glutamate-mediated neurotoxicity in nerve cells.

The inventors of the present invention examined which ginsenosides inhibit the toxicity of glutamate and confirmed that 20(S)-ginsenoside Rg₃,20(S)-ginsenoside Rh₂, that is, a metabolite of ginsenoside Rg₃, and the mixture thereof have excellent inhibition properties against the NMDA receptor, a subtype of glutamate receptors.

Accordingly, the present invention provides a composition for inhibiting glutamate-mediated neurotoxicity, particularly a composition for inhibiting glutamate-mediated neurotoxicity comprising a ginsenoside selected from a group consisting of 20(S)-ginsenoside Rg₃,20(S)-ginsenoside Rh₂ and a mixture of 20(S)-ginsenoside Rg₃ and 20(S)-ginsenoside Rh₂.

Glutamate-mediated neurotoxicity through the NMDA receptor induces nerve cell death during cerebral trauma, cerebral apoplexy and cerebral hemorrhage, which results in irreversible damages to cerebral nerves (Skaper S. D. et al., Ann N Y Acad Sci. 939, 11-22, 2001, and Massieu L. & Garcia O. Neurobiology (Bp) 6, 99-108, 1998). The composition of the present invention, which is capable of inhibiting glutamate-mediated neurotoxicity, can be used for the treatment of nervous diseases including cerebral trauma, cerebral apoplexy, cerebral ischemia and paralysis, and cerebral diseases such as epilepsy (caused by excessive activation of glutamate receptors), Alzheimer's disease and Huntington's disease.

The present invention elucidates that 20(S)-ginsenoside Rg₃,20(S)-ginsenoside Rh₂ and the mixture of 20(S)-ginsenoside Rg₃ and 20(S)-ginsenoside Rh₂ have excellent inhibition efficacy as specific inhibitors of NMDA receptors. Prior research, most closely related to the present invention, discloses that ginsenosides Rb₁ and Rg₃ inhibit nerve cell death in cultured cortical cells (see Kim, Y. C. et al., J. Neurosci. Res. 53, 426-432, 1998). This research tested only the effect of a mixture of 20(S)-ginsenoside Rg₃ and 20(R)-ginsenoside Rg₃, without proposing the inhibition property of 20(S)-ginsenoside Rg₃ against the glutamate-mediated neurotoxicity as disclosed in the present invention.

### BRIEF DESCRIPTION OF DRAWINGS

The above and other objects and features of the present invention will become apparent from the following description of the preferred embodiments given in conjunction with the accompanying drawing, wherein:

FIG. 1A is a graph showing NMDA receptor activity measured by a Ca²⁺-imaging technique, and FIG. 1B is a graph showing NMDA receptor activity determined in accordance with the same technique conducted in FIG. 1A by using ginseng total saponin (GTS) and a known NMDA receptor antagonist, D-APV (D(−)-2-amino-5-phosphono-pentanoic acid)).

FIGS. 2A to 2D are graphs showing results identifying the most superior component among 10 kinds of main ginsenoside components of ginseng, which are screened according to the aforementioned method in FIG. 1, through a NMDA receptor-mediated influx current determined by means of a whole-cell patch-clamp.

FIG. 3 is a graph illustrating nerve cell protection effect of ginsenoside Rg₃ according to the 3-(4,5-dimethylthiazole-2-yl)-2,5-diphenyltetrazolium bromide (MTT) assay.

FIG. 4 shows a decomposition mechanism of 20(S)-ginsenoside metabolized by microorganisms existing in a living body.

FIG. 5 is a graph showing the lowering effect of the NMDA receptor-mediated calcium ion influx of the 9 kinds of 20(S)-ginsenoside shown in FIG. 4.

FIG. 6 is a graph showing the mixture of 20(S)-ginsenoside Rg₃ and 20(S)-ginsenoside Rh₂ having a superior NMDA receptor inhibition effect to the single ginsenoside component.

### DETAILED DESCRIPTION OF THE PRESENT INVENTION

The present invention is described in detail below.

An early stage mechanism of glutamate-induced neurotoxicity primarily shown in brain damages caused by external impacts or cerebrovascular occlusion-induced brain damages such as cerebral infarction, cerebral hemorrhage and paralysis, is investigated using 10 kinds of main ginsenosides of ginseng. The examination system uses a white mouse's hippocampus nerve cells, which are isolated when the mouse is a 18-day-old fetus to a 1-day-old newborn mouse and then cultured in vitro for at least a week, since the hippocampus nerve cell is the most important part for learning and memory models, and nerve cell death is first observed in various pathological phenomena of nerve cells.

Furthermore, glutamate, which is a representative neurotransmitter in a nerve cell, functions through three receptors—NMDA receptors, non-NMDA receptors and metabolic glutamate receptors. An NMDA receptor that induces an excessive amount of calcium influx into the cell is most important for glutamate-induced neurotoxicity. Thus, Ca²⁺ influx caused in NMDA receptor activation is investigated by an intracellular Ca²⁺-imaging technique. Furthermore, the early stage mechanism of NMDA receptor-mediated ion influx is investigated by a whole-cell patch-clamp technique, and the results are confirmed according to a conventional neurotoxicity analysis method, “MTT assay.”

The present invention elucidates that 20(S)-ginsenoside Rg₃,20(S)-ginsenoside Rh₂ and the mixture of 20(S)-ginsenoside Rg₃ and 20(S)-ginsenoside Rh₂ effectively inhibit NMDA receptor-mediated calcium influx.

Therefore, the composition of the present invention for inhibiting glutamate-mediated neurotoxicity comprising a ginsenoside selected from a group consisting of 20(S)-ginsenoside Rg₃,20(S)-ginsenoside Rh₂ and a mixture of 20(S)-ginsenoside Rg₃ and 20(S)-ginsenoside Rh₂ can be effectively used for the treatment of nervous diseases including cerebral trauma, cerebral apoplexy, cerebral ischemia and paralysis, and cerebral diseases such as epilepsy (caused by excessive activation of glutamate receptors), Alzheimer's disease and Huntington's disease.

The composition of the invention can be prepared in various forms including unit dose form for oral administration, injections and syrups, etc.

A preparation suitable for oral administration includes soft and hard capsules, tablets and powders. Such preparation can include one or more pharmacologically inactivated conventional carriers, for example, excipients such as starch, lactose and kaolin; binding agents such as water, gelatin, Arabic gum and tragacanth rubber; disintegrants such as starch and sodium alginate; and lubricants such as talc, stearic acid, magnesium stearate and liquid paraffin.

The composition can also be administered in the form of inhalation drugs, sublingual tablets, topical injections, topical ointments, intramuscular injections, subcutaneous injections and intracutaneous injections.

The composition of the present invention can be formulated in a form of preparation for intravenous injection according to conventional methods. For example, lyophilized crystals are dissolved in a physiological saline solution, distilled water or phosphate buffer solution. The composition can also be prepared as fat emulsion or liposome preparation.

Daily dose of the composition varies depending on various factors including age and health conditions of the subject to be treated. An average adult is given 5˜1000 mg, preferably 10˜500 mg, and more preferably 50˜500 mg of active components once or twice daily. Other doses can be used at the discretion of the physician.

It is obvious to those skilled in the art that the composition is safe for the human body because ginsenosides contained in the present composition are extracted from ginseng used as a herb medicine.

The present invention is described in detail below with reference to the following examples, which are not intended to limit the scope of the invention.

### EXAMPLES

### Example 1

Separation and Culturing Method of Hippocampus Nerve Cell

Hippocampus nerve tissues were separated from an 18-day-old fetus to 1-day-old newborn white mouse using a dissection microscope. The tissues were treated with Leibovitz L-15 medium (Gibco, Grand Island, N.Y.) including 0.05% papain at 37° C. for 20 minutes. Then, the solution was washed with Ca²⁺ or Mg²⁺ free Hank's Balanced Salt Solution (Gibco, Grand Island, N.Y.) several times, and the single hippocampus nerve cell was separated mechanically by adjusting the pore size of the Pasteur pipette. The cell was cultured on a cover slip that was treated with poly-L-lysine in a concentration of 1.5×10⁵ cell/ml.

The cell was cultured in a culturing solution of Neurobasla/B27 (Gibco, Grand Island, N.Y.) to which 0.5 mM L-glutamine, 25 μM glutamate, 25 μM 2-mercaptoethanol, 100 U/ml penicillin and 100 μg/ml streptomycin were added. Half of the medium was replaced twice a week with a medium having no added glutamate. Cells that were cultured for 7 to 15 days were used in the experiment.

### Example 2

Determination of Intracellular Ca Ion Concentration

Fura-2/AM (Molecular probes: Eugene, Oreg.) of the acetoxymethyl-ester form was used as a fluorescent Ca²⁺ label. The hippocampus nerve cell was primarily incubated with 5 μM fura-2/AM and 0.001% Pluronic F-127 in HEPES buffer solution (unit mM: 150 NaCl, 5 KCl, 1 MgCl₂,2 CaCl₂,10 HEPES, 10 glucose, pH 7.4) at room temperature for 40 to 60 minutes and then washed with the HEPES buffer solution several times. After stabilizing the cell for 10 minutes, the change in intracellular Ca ion concentration was determined using an inverted microscope.

Specifically, the cell was exposed selectively to the excitation wavelengths (340 nm and 380 nm) from a xenon arc lamp by a computer-controlled filter wheel (Sutter Instruments, CA). Data was obtained every 2 seconds and a shutter was placed at the light-exposed portion so as to prevent any photo-toxicity on the cell. Emitter fluorescence light, which passed through a 515 nm long-pass filter, reached a cooling CCD camera. The ratios of intensity (340/380 nm) were converted to intracellular free Ca²⁺ concentration ([Ca²⁺]ᵢ) by digital fluorescence analyzer. Analysis of all image data was performed using Universal Imaging software (West Chester, Pa.).

### Example 3

Determination of NMDA Receptor-mediated Membrane Current Using Electrophysiological Method

Whole-cell voltage-clamp recordation was carried out using a perforated patch-clamp method. After a gigaohm seal was formed between the membrane of the cell and the electrode at room temperature to block leakage of the current outward the membrane, the membrane potential was fixed. After 10 minutes, the membrane current was determined. The resistance of the patch electrode was maintained at 3-4 MΩ, and the internal solution of 120 potassium gluconate, 20 NaCl, 2 MgCl₂,10 HEPES, pH 7.4 (unit: mM) was added.

As a solution that allows only the monovalent ion to pass the membrane to produce an electric current, Nystatin stock solution of 25 mg/ml was diluted into a concentration of 250 μg/ml and added to the internal solution. Glycine of 0.001 mM was added to a solution to determine the membrane current (unit: mM, 150 NaCl, 5 KCl, 2CaCl₂,10 HEPES, 10 glucose, pH 7.4) for NMDA receptor activity, and Mg²⁺ was removed therefrom. The membrane voltage was recorded using an EPC-9 amplifier and Pulse/Pulsefit software (HEKA, Germany).

### Example 4

Screening of NMDA Receptor Inhibitor Using NMDA Receptor-Mediated Ca²⁺ Influx

Method for screening NMDA receptor inhibitor via NMDA receptor activity in a hippocampus nerve cell was performed by a Ca²⁺-imaging technique using a Ca-dependent dye, fura-2/AM, as explained in Example 2.

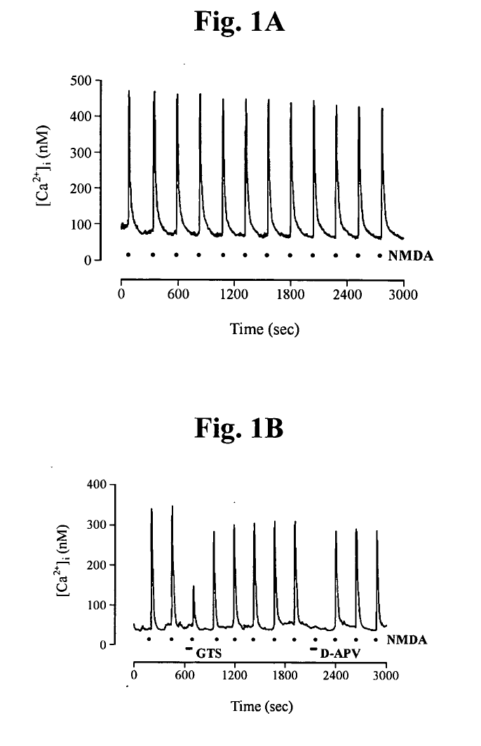

In the short term, whenever NMDA was added, NMDA receptor activation caused a Ca²⁺ influx into the cell. Therefore, after adding NMDA to the cell prepared in Example 1, the Ca2+ influx was observed at least 12 times every 4 to 5 minutes. As a result, the Ca²⁺ concentration was significantly decreased when the constant value of [Ca²⁺]ᵢ (362.6 nM for first and 356.8 nM for 12th application of NMDA) was maintained without desensitization and a known NMDA receptor antagonist, D-APV and ginseng total saponin (GTS) were treated to the cell (FIG. 1). Therefore, the system was used in the NMDA receptor inhibitor screening of ginseng components.

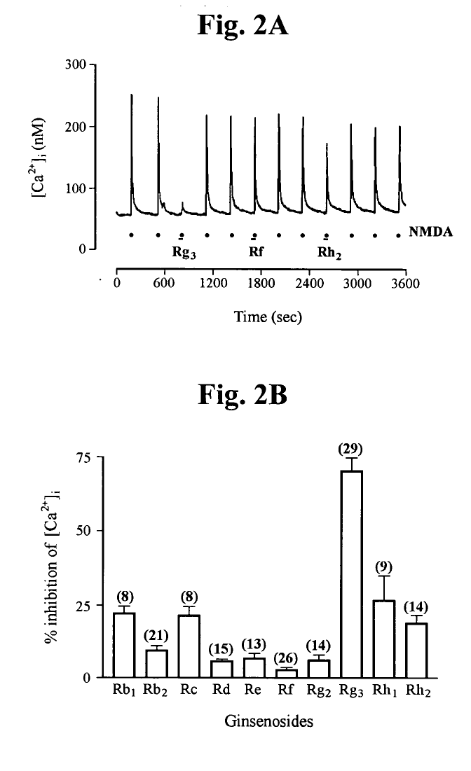

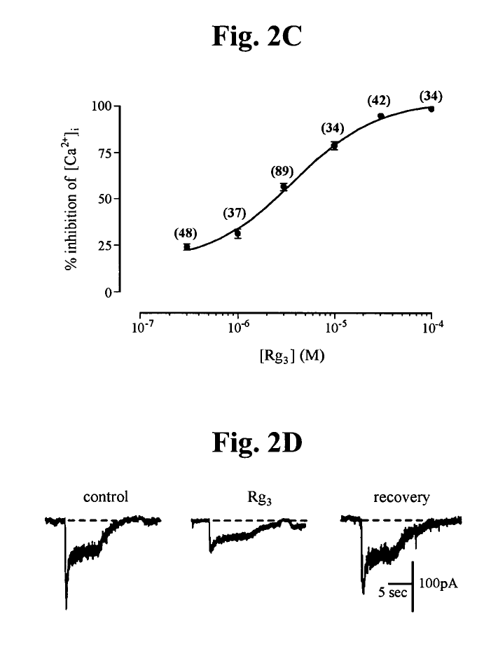

Among main components of ginseng, 10 ginsenosides (ginsenoside-Rb₁, -Rb₂, -Rc, -Rd, -Re, -Rf, -Rg₂, -Rg₃, -Rh₁ and -Rh₂) of a state including isomers3 of C-20 position were screened. The NMDA receptor inhibition efficiency of the ginsenoside Rg₃ was shown to be superior to those of other ginsenosides (FIGS. 2A and 2B).

The NMDA receptor inhibition efficiency of the ginsenoside Rg₃ was also confirmed by the electrophysiological whole-cell patch-clamp method as described in Example 3, which could directly determine NMDA receptor-mediated current and calcium influx (FIG. 2D). It also showed that the efficiency of ginsenoside Rg₃ was dependent on its concentration showing an IC₅₀ value of 3.8 μM (FIG. 2C).

As stated above, the experiments of FIGS. 2A to 2C were to determine Ca²⁺ influx caused by NMDA activity, when NMDA was administered to the cultured hippocampus nerve cell (5˜10 seconds), by using the Ca²⁺-imaging technique. Meanwhile, FIG. 2D shows that ginsenoside Rg₃ inhibits the inward current that was produced by an influx of NA⁺ ions, etc., into the cell by NMDA receptor activity in addition to the Ca²⁺ ion influx. The results of FIG. 2 suggest that ginsenoside Rg₃ shows its inhibition action by blocking the influx of ions that are elicited by NMDA receptor activation. The used amounts of ginsenosides are 10 μM except in the concentration dependent experiments.

3-(4,5-dimethylthiazole-2-yl)-2,5-diphenyltetrazolium bromide (MTT) assay was carried out to determine the survival rate of cells against cytotoxicity by NMDA in nerve cells. Primarily cultured white mouse hippocampus nerve cells were cultured in a 24-well plate, to which a Neurobasal/B27 (Gibco, Grand Island N.Y.) medium including 0.5 mM L-glutamine, 25 μM glutamate, 25 μM 2-mercaptoethanol, 100 U/ml penicillin and 100 μg/ml streptomycin was added for 6-14 days. The cells were pretreated with 100 μg/ml GTS and 10 μM Rg₃ using a Mg²⁺ free HEPES buffered solution including 1 μM glycine for 1 minute and then treated with NMDA for 10 minutes. Thereafter, the medium was exchanged with the normal culture medium. Twenty-four hours after such treatment, the cells were treated directly with a 50 μl MTT solution in phosphate buffered saline (PBS) (1 mg/ml). Four hours after MTT treatment, the supernatant was removed, and the produced formazan was resolved in 100 μl dimethylsulfoxide (DMSO). Optical density at 560 nm was read using an automated spectrophotometric plate reader.

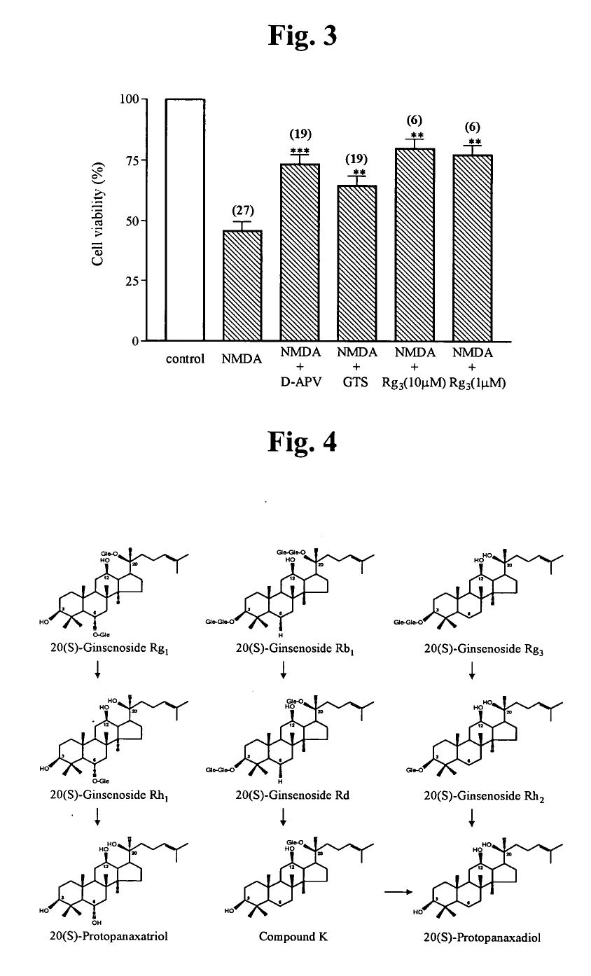

The MTT assay demonstrated that D-APV (a NMDA receptor antagonist), ginseng total saponin (GTS) and ginsenoside Rg₃ effectively increase cell viability against neurotoxicity caused by NMDA (FIG. 3).

Although 30 or more types of ginsenosides are present in ginseng, many ginsenosides among them are produced in vivo metabolism of their precursors (FIG. 4). As such, the efficacy on the NMDA receptor was also investigated for ginsenoside metabolites produced by microorganisms in the stomachs of humans, especially pure compounds of (S)-form at C-20 position.

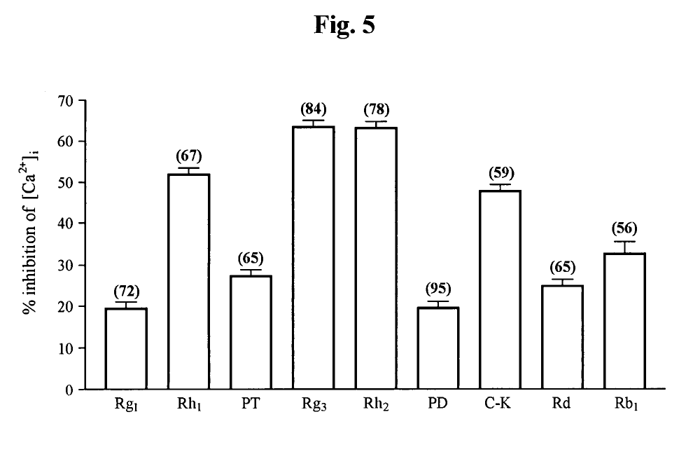

For screening the pure compounds of (S)-form at C-20 position, the experiments were performed as shown in FIGS. 2A to 2C using 20(S)-ginsenoside-Rb₁, -Rd, -Rg₁, -Rg₃, -Rh₁, -Rh₂,20(S)-protopanaxadiol, 20(S)-protopanaxatriol and compound K. As a result, 20(S)-ginsenoside Rg₃ and 20(S)-ginsenoside Rh₂ show the most excellent inhibition effect for NMDA receptor activity (FIG. 5).

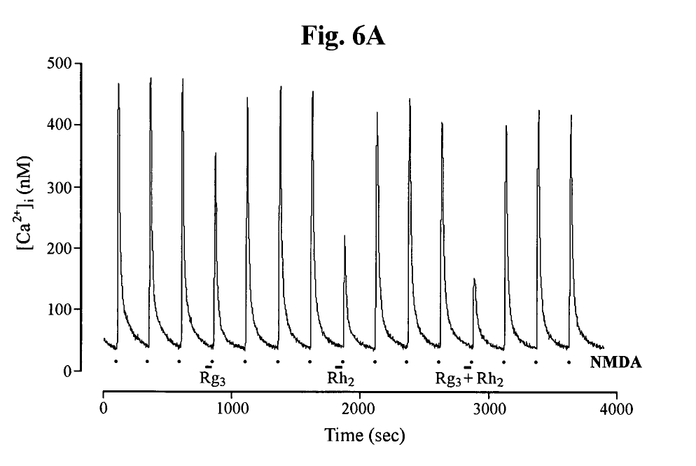

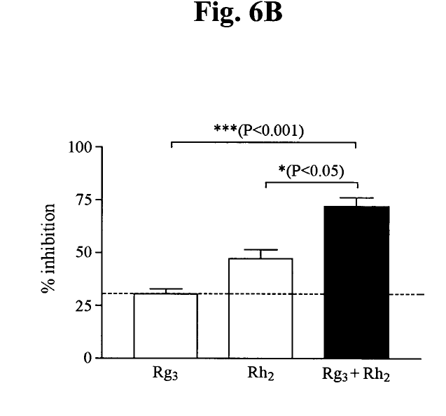

The effect of the mixture of 20(S)-ginsenoside Rg₃ and 20(S)-ginsenoside Rh₂ was determined by mixing 1 μM 20(S)-ginsenoside Rg₃ and 1 μM 20(S)-ginsenoside Rh₂ at a ratio of 1:1 and performing the experiments as shown in FIGS. 2A to 2C. As a result, the mixture shows a NMDA receptor inhibition effect superior to the single ginsenoside as shown in FIG. 6 (FIG. 6A shows results obtained from a single cell, FIG. 6B shows results obtained from several cells and a statistical significance (n>9)).

Table 1 indicates the NMDA receptor-mediated Ca²⁺ influx inhibition of GTS, ginsenoside Rg₃ and ginsenoside Rh₂ in a mixture form of 20(S)-form and 20(R)-form, 20(S)-ginsenoside Rg₃ and 20(S)-ginsenoside Rh₂. Ginsenoside Rg₃ shows an equivalent efficacy regardless of an S-form or R-form isomer and their mixture. In the case of ginsenoside Rh₂, 20(S)-ginsenoside Rh₂ shows a superior effect to the mixture of S-form and R-form isomers.

TABLE 1

| | | % inhibition of NMDA |
|---|---|---|
| | | receptor-mediated |
| | Concentration | [Ca²⁺]i infulx¹ |
| Ginseng total saponin (GTS) | 100 μg/ml | 62.0 ± 2.1 |
| ginsenoside Rg₃ | 1 μM | 31.2 ± 2.5 |
| (mixture of 20(S)-form and | | |
| 20(R)-form) | | |
| 20(S)-ginsenoside Rg₃ | 1 μM | 30.4 ± 2.3 |
| ginsenoside Rh₂ | 10 μM | 18.6 ± 2.7*** |
| (mixture of 20(S)-form and | | |
| 20(R)-form) | | |
| 20(S)-gensenoside Rh₂ | 1 μM | 47.1 ± 4.3 |
| mixture of 20(S)-ginsenoside | 1 μM | 71.9 ± 4.2 |
| Rg₃ and 20(S)-ginsenoside | | |
| Rh₂ | | |

¹tested in the cultured mouse hippocampus nerve cell,
***P < 0.001, compared to the determined value of 20(S)-ginsenoside Rh₂, tested at 10 μ M due to very low efficacy at 1 μ M.

The present invention is to elucidate the action mechanism of 20(S)-ginsenoside Rg₃,20(S)-ginsenoside Rh₂ and their mixture on a nerve cell and on their efficacy. Since the composition of the invention has an excellent neurotoxicity inhibition effect and nerve cell protection effect, it can be used in treating encephalopathies such as cerebral trauma, cerebral apoplexy, cerebral ischemia, paralysis, epilepsy, Alzheimer's disease and Huntington's disease.

What is claimed is:

1. A composition for inhibiting glutamate-mediated neurotoxicity comprising a ginsenoside selected from a group consisting of 20(S)-ginsenoside Rg₃,20(S)-ginsenoside Rh₂ and mixture of 20(S)-ginsenoside Rg₃ and 20(S)-ginsenoside Rh₂.

2. The composition of claim 1, wherein the ginsenoside is 20(S)-ginsenoside Rh₂.

3. The composition of claim 1, wherein the ginsenoside is the mixture of 20(S)-ginsenoside Rg₃ and 20(S)-ginsenoside Rh₂.

4. The composition of claim 1, wherein the composition is used for treating cerebral diseases selected from a group consisting of cerebral trauma, cerebral apoplexy, cerebral hemorrhage, paralysis, epilepsy, Alzheimer's disease or Huntington's disease.
